(19) United States
(12) Patent Application Publication
(10) Pub. No.: US 2002/0142354 A1
(43) Pub. Date: Oct. 3, 2002

(54) Method of protein analysis

(76) Inventors: Sophie Elizabeth Victoria Wildsmith, Hertford (GB); Rhiannon Yallop, Hoddesdon (GB); Peter John Bugelski, Pottstown, PA (US); David Gwyn Morgan, Wayne, PA (US)

Correspondence Address: GLAXOSMITHKLINE, Corporate Intellectual Property - UW2220, P.O. Box 1539, King of Prussia, PA, 19406-0939, US

(21) Appl. No.: 10/056,184

(22) Filed: Jan. 24, 2002

Related U.S. Application Data
(62) Division of application No. 09/744,823, filed on May 29, 2001, now abandoned.
Application No. 09/744,823 is a 371 of international application No. PCT/GB99/02478, filed on Jul. 29, 1999.

(30) Foreign Application Priority Data
Jul. 29, 1998 (GB) 9816514.5

(51) Int. Cl. G01N 33/53

(52) U.S. Cl. 435/7.2

(57) ABSTRACT

A method for analyzing the protein or peptide content of a mixture comprising exposing a spatial array of protein recognition units to the protein or peptide containing mixture, and detecting the binding of proteins or peptides to the protein recognition units.

The invention relates to a method for the rapid analysis of the protein or peptide composition of a mixture, in particular for analyzing an extract derived from tissues or cells in order to measure the differential expression or abundance of proteins. The invention can be used in the analysis of the temporal and spatial changes in protein expression in response to drug treatments, particularly when investigating drug induced toxicity.

Proteins and peptides play a central role in many cellular processes in all cellular biosystems. Examples of such functions include structural and catalytic roles. The ‘proteome’, that is the protein expression pattern in a given tissue or cell type, is a notional reflection of the dynamic nature of these cellular systems. Genes are constantly being induced or switched off to modulate the levels of different proteins within the cell and these proteins are sometimes modified or secreted from the cell to act as signals to other cells. These signals in turn alter the gene expression pattern of the receiving cell. These dynamic systems, despite being very tightly controlled are, however, inherently susceptible to disturbance. Bacterial or viral infection, for example, results in a change in this equilibrium as the system attempts to repel the invading micro-organisms and restore the system to normal. The delicate balance of cellular processes can also be disturbed as a result of genetic abnormalities. Similarly, environmental insults such as physiological trauma and exposure to chemicals can alter the proteome of affected tissues and cells. Such disturbance of the qualitative, quantitative, temporal and spatial distribution of proteins and peptides are frequently associated with serious cellular dysfunction and can lead to tissue damage and in some cases death.

The disturbances of cellular processes, as reflected in the proteome of the cell or tissue are of particular concern in the pharmaceutical industry in the safety assessment of new drugs. For example ingestion of a novel chemical compound is an environmental insult. Many new drugs are identified by screening large collections of chemical compounds for a compound that demonstrates an ability to block an interaction between two components of a cellular process. In a patient suffering from a condition where this interaction is unwanted or is excessive, due to abnormally elevated levels of one of the components, reducing the levels of this interaction by treating the patient with a blocking drug will restore the system to equilibrium. Therefore, by their very nature, many drugs will affect the balance of proteins in the cell or tissue. However many drugs, in addition to effecting the desired activity upon a cellular process, also have unwanted effects on other cellular processes (so called ‘side effects’), and this will be reflected in the change in the proteome. In determining the safety of novel compounds it is necessary to examine and compare the proteomes from treated and untreated animals, tissues or cells, and identify any changes that have taken place as a result of administering the new drug. It would also be an advantage to be able to identify the particular proteins whose expression is affected by the treatment.

Detection of proteomic changes is not a trivial task despite the existence of several protein analysis techniques. Existing techniques fall into the following categories:

1) Quantitative, non-specific, total protein: Lowry, Bradford, Coomassie, BCA assays, for quantitation of proteins in solution.

2) Qualitative (can be made quantitative): staining of gels with silver stain, Coomassie stain, radioactivity and fluorescent staining.

3) Quantitative and specific: e.g. immunological methods such as detection of antigens by ELISA, immunoprecipitation and Western blots.

4) Qualitative and quantitative (non-specific): high pressure liquid chromatography (hplc), fast protein chromatography (fpc), capillary zone electrophoresis (cze).

5) Qualitative, high accuracy: mass spectrometric methods e.g. (MALDITOF).

One technique which is currently used is 2-Dimensional (2-D) gel electrophoresis. However this technique is laborious, requires skilled staff, is not easily reproducible and the analysis of data is complex. Proteins, which are extracted from a tissue or cells, are separated in a gel in one dimension based upon their isoelectric point. They are further separated by electrophoresis, in a second dimension, on the basis of their molecular weight. The gels are stained, using either conventional protein stains, such as Coomassie blue or silver stain, or by more sensitive fluorescent stains. The result is a slab of gel, usually approximately 20 cm ×30 cm with a multitude of “spots”. This is scanned using the appropriate wavelength of light (see for example Anderson et al, Tox. Path., 1996, 24, 72-76). Complex software is required to locate spots, differentiate between discrete spots and artifacts and to identify the proteins of interest. Gels from different samples can be compared, e.g. from treated and untreated tissues, but problems can arise in locating identical spots due to distortion of the gel and other reproducibility problems. Not every protein in a sample will be identified using this technique-only a certain pH range can be examined at any one time and there will be a limit to the size range which can be looked at on one gel. Moreover some proteins do not stain with certain dyes.

None of the currently available methods enable a high throughput, qualitative and quantitative analysis of proteomic change. The present invention provides a method for analyzing the protein or peptide content of a mixture which enables a high throughput, qualitative and quantitative analysis of proteomic change.

According to the invention there is provided a method for analyzing the protein or peptide content of a mixture comprising exposing a spatial array of protein recognition units to the protein or peptide containing mixture, and detecting the binding of proteins or peptides to the protein recognition units.

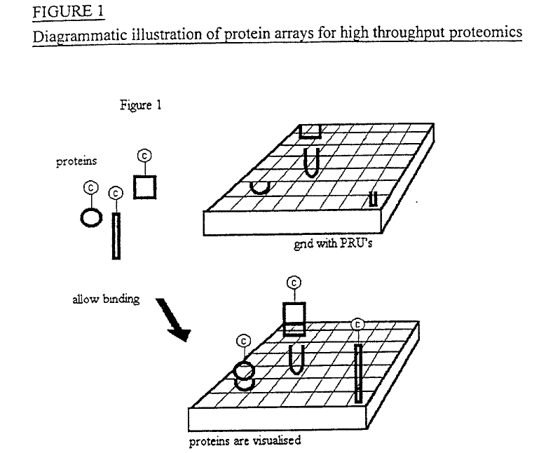

FIG. 1 shows a diagrammatic representation of the method of the invention. Protein recognition units (PRU's) are immobilized or synthesized on a grid system. Four PRU's are shown, but the number of PRU's may vary depending on the application e.g. low density (<100 PRU/cm²) or high density (>100 PRU/cm²). Proteins are recognized by, and bind to, the PRU's. The proteins are pre- or post-labelled so that a signal from a given grid co-ordinate indicates that the protein which corresponds to that PRU is present.

The spatial array of PRUs preferably comprises a plurality of different PRUs. The PRUs are preferably immobilized on a solid or semi-solid support. The PRUs are preferably arranged in a specific, coordinated pattern, e.g. in a grid format, such that the location of each PRU is known. The PRUs may be minimal recognition units (MRUs), antibodies or antibody fragments, high affinity peptides or complementarity determining regions (CDRs). The PRUs may be of known or unknown sequence content i.e. the identity of the PRU or bound protein can be determined retrospectively, if desired, by releasing the PRU from the solid surface, separating the bound protein and sequencing both.

The PRUs are preferably CDRs. In native IgG antibodies the variable heavy and light chains each contribute three regions called CDR's which are responsible for binding antigen. Each region is typically 7-20 amino acids long, e.g. 15-17 amino acids, and its sequence defines the specificity and affinity of that CDR for the antigen. There is increasing evidence that these regions of CDR-peptides are capable of autonomous specific binding to antigens, see for example Steinbergs J, Kilchewska K, Lazdina U, Dishlers A, Ose V, Sallberg M, Tsimanis A, Hum. Antibod. Hybridomas, 1996, 7, 3; William W V, Kieber-emmons T, VonFeldt J, Greene M I, Weiner D B, J. Biol. Chem., 1991, 266, 5182; Saragovi H U, Fitzpatrick D, Raktabutr A, Nakanishi H, Kahn M, Greene M, Science, 1991, 253, 792, Welling W G, van Gorkum J, Damhof R A, Drijhout J W, Bloemhoff W, Welling-Wester S, J. Chromatogr., 1991, 548, 235; and Levi M, Sallberg M, Ruden U, Herlyn D, Maruyama H, Wigzell H, Marks J, Wahren B, Proc. Natl. Acad. Sci. USA., 1993, 90, 4373; and hence they have been proposed as new generation antibody fragments with reduced immunogenicity and as anti-viral molecules, see Sivolapenko G B, Douli V, Pectasides D, Skarlos D, Sirmalis G, Hussain R, Cook J, Courtney-Luck N S, Merkouri E, Konstantinides K, Epenetos A A, Lancet 1995, 346, 1662; and Rossenu S, Dewitte D, Vandekerckhove J, Ampe C, Journal of Protein Chemistry, 1997, 16, No.5.

Antibody fragments may, for example, be generated using phage display technologies. CDRs can be produced by recombinant means or can be chemically synthesized. CDRs can be chemically synthesized according to known CDR sequences using either standard protein synthesis or using a combinatorial synthesis approach.

Spatial arrays of CDRs are novel, thus according to a further aspect of the invention there is provided a spatial array of CDRs for use in the method of the invention.

CDR sequences may be determined by analysis of various numbering systems such as the Kabat, Chothia, AbM and Contact numbering schemes:

The Kabat definition is based on sequence variability and is the most commonly used.

The Chothia definition is based on the location of the structural loop regions.

The AbM definition is a compromise between the two used by Oxford Molecular's AbM antibody modeling software.

The Contact definition is specified by University College London and is based on an analysis of the available complex crystal structures. Mean contact data is produced by an analysis of the contacts between antibody and antigen in the complex structures available in the Protein Databank (MacCallum, R. M., Martin, A. C. R. and Thornton, J. T., J. Mol. Biol., 262, 732-745). Briefly, the number of contacts made at each position, defining contact as burial by >1 square Angstrom change in solvent accessibility. These data give a simple measure of how likely a residue is to be involved in antigen contact.

H3 and L3 make the most contacts with their antigen because they are central in the combining site. H1, L1 and H2 make fairly similar numbers of contacts being rather more peripheral with L2 making the fewest contacts overall. Topographically it is in an equivalent position to H2 (the light and heavy chains have a rotational axis of pseudo-symmetry), but it is much shorter so has less chance of reaching the antigen. L3 makes a lot of contacts but they tend to be generally “sticky” rather than specific. The following table lists the positions of the CDRs according to each numbering system. “L” refers to “light chain” and “H” to “Heavy chain” and the numbers correspond to amino acid sequences.

Table of CDR Definitions

| Loop | Kabat | AbM | Chothia | Contact |
|---|---|---|---|---|
| L1 | L24-L34 | L24-L34 | L24-L34 | L30-L36 |
| L2 | L50-L56 | L50-L56 | L50-L56 | L46-L55 |
| L3 | L89-L97 | L89-L97 | L89-L97 | L89-L96 |
| H1 | H31-H35 | H26-H35 | H26-H32/34 | H30-H35 |
| H2 | H50-H65 | H50-H58 | H52-H56 | H47-H58 |
| H3 | H95-H102 | H95-H102 | H95-H102 | H93-H101 |

CDR sequences can be found by using one of, or a combination of the above mentioned methods. A few amino acids either side of the CDR sequence can also be used as these can also enhance binding. The methods may be applied to antibody light and heavy chain protein sequences from any of protein search sites e.g. Entrez, Swiss-Prot. If the protein sequence for a desired antibody is not available the nucleotide sequence can be translated into peptide sequence by using a package such as Lasergene. (Dnastar—www.Dnastar.com.)

In operation the spatial array of PRUs is exposed to the protein or peptide containing mixture, e.g. as a tissue or cell extract, under conditions favourable to protein binding. Proteins or peptides in an extract or mixture are subsequently bound to the grid in a specific manner. The binding of proteins or peptides to the PRUs may be effected by visualization of the proteins or peptides e.g. by staining or other methods such as radiolabelling or chemical labeling, either before or after the binding step. Biotinylation, fluorescence, chemiluminescence or other labeling techniques may also be used. Analysis of the resultant binding pattern may be undertaken using an appropriate detection system such as optical or laser scanning technology and a comparison of results between, for example, drug treated and untreated control tissues can be undertaken using suitable computer software.

The protein or peptide containing mixture that is analyzed according to the method of the invention is preferably a cell extract isolated from a biological organism, e.g. from mammalian tissue. The cell extract is more preferably from a mammal treated with a chemical compound of therapeutic potential.

The method of the invention provides a fast, reproducible and robust alternative to the methods currently used for determining the protein expression pattern of tissues or cell types, i.e. proteomics. The method may be applied to proteomics in, for example, toxicology, clinical trials, process monitoring, biotechnology and pharmaceutics, either as a research or diagnostic tool. The method may be used for analyzing the spatial and temporal changes in the expression of proteins in mammalian tissues and cell types when exposed to environmental stimuli such as pharmaceutical reagents, as well as other environmental stimuli or factors. The method allows for the comparison of targets treated with therapeutic drugs and untreated controls to provide a quantitative and/or semi-qualitative analysis of the proteome of the treated and untreated (control) target systems e.g. cells, tissues or biological organisms. The method may also be adapted for use in, for example, drug manufacturing, purity analysis (e.g. in food and agriculture) and for diagnostics, e.g. for detecting proteins in urine or blood.

Thus the invention also provides a method of determining the effect of an environmental factor on protein expression in a target cell, tissue or biological organism, said method comprising the steps of:

(a) providing a plurality of identical spatial arrays of protein recognition units;

(b) exposing the target to an environmental factor for a sufficient time to affect protein expression in said target;

(c) extracting and isolating a protein or peptide containing mixture from said exposed target of step (b);

(d) extracting and isolating a control protein or peptide containing mixture from a target not exposed to said environmental factor;

(e) exposing a spatial array of protein recognition units to the protein or peptide containing mixture from the exposed target, and detecting the binding of proteins or peptides to the protein recognition units;

(f) exposing a spatial array of protein recognition units to the protein or peptide containing mixture from the control target, and detecting the binding of proteins or peptides to the protein recognition units; and

(g) comparing the first and second binding patterns detected in steps (e) and (f) to identify any change in said first pattern from the control pattern, indicative of an effect on protein expression in the target exposed to said environmental factor.

The terms environmental factor as used herein describes a wide variety of physical, chemical or biological factors which may cause changes in protein expression in a target, e.g. cell, tissue or biological organism, when exposed to that factor. For example, physical environmental stimuli can include, without limitation, diet, an increase or decrease in temperature, an increase or decrease in exposure to ionizing or ultraviolet radiation, and the like. Biological/chemical stimuli can include, without limitation, administering a transgene to the target, or eliminating a gene from the target; administering an exogenous synthetic compound or exogenous agent or an endogenous compound, agent or analog thereof to the target.

As an example, an exogenous synthetic compound can be e.g. a pharmaceutical compound, a toxic compound, a protein, a peptide, or a chemical composition. An exogenous agent can include natural pathogens, such as microbial agents, which can alter protein expression. Examples of pathogens include bacteria, viruses, and lower eukaryotic cells such as fungi, yeast, molds and simple multicellular organisms, which are capable of infecting a target and replicating its nucleic acid sequences e.g. in the cells or tissue of that target. Such a pathogen is generally associated with a disease condition in the infected mammal.

An endogenous compound is a compound which occurs naturally in the body. Examples include hormones, enzymes, receptors, ligands, and the like. An analogue is an endogenous compound which is preferably produced by recombinant techniques and which differs from said naturally occurring endogenous compound in some way.

The invention also provides a method of predicting the toxic effect of a selected test compound on a mammalian subject, said method comprising the steps of:

(a) performing the method of claim 2 or 3 by calibrating the spatial arrays of protein recognition units with a plurality of known and structurally distinct toxicants having a common known toxic effect on mammalian subjects and generating a common “fingerprint” binding pattern characteristic of said common toxic effect;

(b) screening a test compound according to the method of claim 2, steps (a)-(e), to generate a test compound binding pattern; and

(c) comparing the binding pattern of the test compound to said fingerprint binding pattern,

wherein substantial identity between the fingerprint and test patterns indicates that said test compound shares said common toxic effect.

All publications, including but not limited to patents and patent applications, cited in this specification are herein incorporated by reference as if each individual publication were specifically and individually indicated to be incorporated by reference herein as though fully set forth.

The invention will now be illustrated by way of the following examples.

### EXAMPLE 1

### Protein Array with CDR's as Protein Recognition Units and Fluorescent Dye Detection

### 1.1 Preparation of Spatial Arrays

CDR's are synthesized according to known peptide synthesis techniques e.g. Sällberg M et al. ImmunoL Lett., 1991, 30, 59. Peptides can be analyzed and purified, for example by reverse phase HPLC as described by Steinbergs et al. Hum. Antibod. Hybridomas, 1996, 7(3),106-112. CDRs can either be synthesized according to known sequences from literature or experimentation or they can be synthesized as unknowns. This can be either a random synthesis or a combinatorial synthesis. For a CDR of 16 amino acids, the theoretical number of all possible permutations exceeds what would be manageable in practice (1.8 ×10⁹ possibilities). This number can be reduced by incorporating knowledge of CDR structures, common motifs and probabilities of sequences into the decision making process. Examples of different approaches are a) removing sequences featuring the same amino acid more than a certain number of times (or in succession), or b) limiting amino acid usage to that similar to the natural or known occurrence in CDR's. An alternative to this informed or “intelligent” combinatorial approach is a pre-screened combinatorial approach. In this case, known proteins of interest are used to pull out corresponding CDR's from a random synthesis and therefore the CDR sequence need not be known.

The CDRs once synthesized can be arrayed by applying them to a support in any format wherein the CDRs are arrayed in a spatial manner. The CDRs are preferably gridded. The support can comprise a gel, plastics, nylon, nitrocellulose, glass, silica, carbon, perfluorocarbons, alumina, ceramics, polymers, cellulose, cross-linked polysaccharides, metals or any other material on which the CDRs can be immobilized. Standard methods for immobilization are well documented (e.g. Mosbach, Methods in Enzmnology, 1976, 44; Immobilized biochemicals and affinity chromatography, advances in experimental medicine and biology, Ed R.Dunlap, Plenum Press, N.Y., 1974; 42; and Chemistry of protein conjugation and cross linking. Wong, CRC Press, 1993) and usually exploit the reactive groups on amino acids such as the amine and thiol groups for disulphide linkages, thioether bonds, hindered disulfide bonds and covalent bonds. Other linkage methods include introducing inert amino acids and utilising photoactivation, metal ion linkages and other linkering/anchoring molecules. Preferred immobilization techniques do not disrupt the CDR properties and do not interfere with the protein recognition attributes of the CDRs.

### 1.2 Application of Test Solution

Once designed and fabricated, the arrays can be used to detect proteins in a mixture. The arrays are flooded with a protein mixture derived from any source, e.g. tissue homogenate, cell free suspension, supernatants. The protein mixture can be altered to facilitate binding. For instance, the proteins can be dialysed into a buffer or a solution with a pH and ionic constitution such as to enable binding. Throughout the binding process, the conditions can be altered in order to affect the stringency of binding. A suitable hybridisation buffer is 0.5M phosphate buffered saline, pH7. In this case, stringency can be decreased by lowering the pH of the solution during the binding process. The binding procedure can be allowed to occur over a period of several hours. The protein arrays can then be washed using buffer as above.

### 1.3 Detection of Protein Binding

The proteins are preconjugated with a fluorescent dye such as Alexa, BODIPY, SYPRO™ Orange or SYPRO™ Red stains from Molecular Probes. Any other fluorescent dyes which bind proteins could be used. The preferred embodiment is a covalent method of attachment such as binding of the primary amine groups (e.g. Alexa. BODIPY dyes). Images of the arrays can be obtained using a laser scanner and CCD acquisition system, for example the STORM™ system from Molecular Dynamics.

An alternative labeling methodology involves radioactive labeling, which may be achieved, for example by adding S³⁵ to the growth medium in which proteins are synthesized. In this way nascent proteins will be radiolabelled. When radiolabelled proteins are bound to the array they can be visualized using standard autoradiography techniques.

Images of the arrays can be used to analyze the proteins present and the quantities of proteins present in the samples. If the CDRs are known, then the position of the CDRs on the array is known and the relative concentrations of those proteins can be determined. Controls of known concentration can be used for quantification purposes. If the CDR's are unknowns (generated using a combinatorial approach) then patterns of expression of proteins can be monitored. If certain proteins prove interesting, for example, if the expression level changes with at different stages of a cell cycle, then those proteins can be isolated and identified.

### EXAMPLE 2

### Protein Array with Bacteriophage Display Proteins as Protein Recognition Units

In this method bacteriophage (hereafter “phage”) expressing heterologous protein fragments or peptides, including random peptides, on their surfaces are immobilized on a support. The heterologous protein fragments are preferably antibody variable regions. The displayed protein fragments or peptides are thereby enabled to function as PRUs in the method of the invention. The phage-antibody display system preferably allows expression of ScFv or Fabs on the surface of a bacteriophage. Antibody variable region genes are cloned into the genes encoding a coat protein, for example a minor coat protein, of the filamentous bacteriophage thereby creating a fusion protein. The antibody genes are selected from a gene repertoire, for example mRNA extracted from B cells, and expressed in the phage library.

The phage display library thus produced can be used to screen for proteins in mixtures. Binding of proteins to phage displayed antibodies can be achieved using conditions similar to those described by Palmer el al, Vox Sanguinis, 1997, 72, 148-161.

### EXAMPLE 3

### Application of Protein Arrays to Monitor Dynamic Changes to Protein Expression Induced by Pharmaceutical Compounds

The invention enables the investigation of changes in protein expression in a tissue or cell-type of interest in response to exposure to a therapeutic compound. This aspect of the invention is facilitated by preparing a series of test animal samples, tissue samples or cell-cultures, introducing the compound and harvesting the cells at selected time points. A cellular extract can then be prepared from the harvested cells using standard cell disruption techniques, in the presence of appropriate protease inhibitors, and the extracted proteins stained with fluorescent stain. The spatial array of PRUs is exposed to the protein extract under conditions allowing proteins in the extract to bind to the PRUs in the array. The array can then be visualized using excitation of the specific wavelength for the fluorophore.

The “pattern” of fluorescence is compared in the treated and untreated cells. Proteins of interest which occur on one array, but not another can be isolated and identified. After some time, the proteins which the PRU's recognize will become known and a database can be constructed which relates the expression of proteins in response to drugs. This database can be used to predict the likely action and toxicity of a compound and possibly it's pharmacology. It will also contribute to an understanding of cascades of events associated with certain drugs.

### EXAMPLE 4

### Proteomics Array to Differentiate Protein Expression in Response to Classic Hepatotoxins

### Example Tissues

The protein expression in response to five classic hepatotoxins is studied using the method of the invention. Antibodies to proteins are used, where the protein expression may differ to the control and a specific pattern of expression can be identified for a given compound. The hepatotoxins used are sodium valproate, erythromycin estolate, methotrexate, acetominophen and coumarin.

### Example PRU's

A number of antibodies, fragments or CDR's are immobilized on a solid support. In practice several thousand PRU's could be laid down. In the simplest case, nine antibodies are bound to a sheet of white polystyrene. The antibodies are from a selection of commercially available antibodies such as those to alkaline phosphatase, GST-glutathione, transferase, TNF, p53, Bcl-2, hepatocyte growth factor, insulin-like growth factor, folic acid (all available from Sigma). In addition, rat whole serum antibodies can be used for control spots.

### Example Method of Immobilization

The antibodies, in coating buffer (e.g. 0.05M carbonate buffer, pH9.6) are spotted onto the polystyrene sheet (PVDF, nitrocellulose or glass could be used) in triplicate and at 3 different concentrations. They are incubated overnight at 4° C. in a humidity chamber to allow binding. The sheet is rinsed and then incubated for one hour with blocking buffer to prevent non-specific absorbtion (0.05M carbonate buffer, pH9.6, 0.5% casein). The sheet is then washed with wash buffer (sodium phosphate buffer)₁ and sample is applied, diluted in phosphate buffered saline to a concentration of 1 mgml₋₁.

1. sodium chloride (58.44 g), disodium hydrogen orthophosphate (11.53 g), sodium dihydrogen orthophosphate dihydrate (2.925 g), EDTA (37.22 g), Tween 20 (2 ml), L-butanol (100 ml), distilled water to 10 L, pH7.

### Example Sample Preparation

The samples are cell lysates, made from the livers of rats treated with the above hepatotoxins and prepared according to the RiboLyser protocol (Hybaid). The cell lysates prepared in sample buffer containing a mixture of protease inhibitors of 2 mM phenylmethylsulfonyl fluoride, 2 ug/ml aprotinin, 2 mM sodium orthovanadate, 100 mM sodium fluoride, 10 mM sodium fluoride, 10 mM sodium pyrophosphate, 10 mM sodium molybdate and 2 mgml⁻¹ leupeptin. The lysates are concentrated using a Pierce KwikSpin ultrafiltration unit with a 5 kDa cut off before determining the total protein concentration using a Coomassie Plus protein assay (Pierce). The semi-purified proteins are conjugated with Texas Red-X (Molecular Probes, product no F-6162) according to the protocol provided by the manufacturer. Texas Red absorbs at 595 nm and emits at 625 nm and thus the resulting sheets can be scanned on a Molecular Dynamics Storm system.

### Example Results

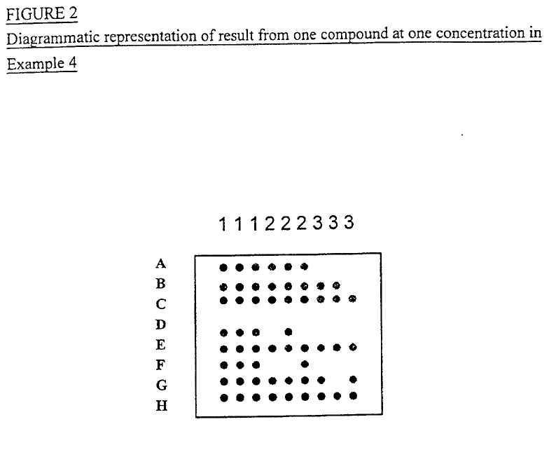

FIG. 2 is a diagrammatic example of the resulting scanned sheet where A to I are PRUs spotted in three concentrations from left to right and the spots indicate the protein present in that sample from that tissue.

### Example Conclusions

These results indicate that no protein D is detected in the sample but there is high expression of protein I. This pattern may enable this compound to be distinguished from the untreated sample and the four other hepatotoxicant treated livers. These results can be used as a pattern matching exercise, and changes in expression can be used for developing hypotheses regarding mechanisms of toxicity. Where a large number of PRUs are used, for example using synthetic CDRs, particularly high or low expressing proteins may be identified and their importance recognized for the first time.

### EXAMPLE 5

### Poteomics Array to Differentiate Protein Expression in the Liver Following Administration of Azathioprine Compared to Positive Control

### 5.1 PRU Immobilization/Grid Design

Coat Silane coated glass slides (Sigma) with glutaraldehyde 2%(Agar Scientific) and leave for 1 hour. Draw an area on the slide with PAP pen (Sigma) this creates a hydrophobic barrier around the area to be spotted with antibodies or protein recognition units. Spot a solution-containing antibody/protein recognition unit directly onto the slide in 0.5 ul volumes, diluted in coating buffer (0.2 M sodium carbonate buffer, pH 9.6 or KPL coating buffer (Kirkegaard and Perry Laboratories)). Leave at 4° C. O/N in a humidified container.

PVDF or Nitro-cellulose slide-Oncyte slides (Grace Biolabs) can also be used as a surface these are prepared differently from the above as follows:

Pre-wet PVDF membrane by floating membrane in 100% methanol (ROMIL) and submerging briefly. Transfer membrane to a container of TBS (no Tween 20) & submerge for 2 minutes. To prevent the membrane drying out during spotting, place membrane on top of 2 sheets of blotting paper wet with TBS. Spot a solution containing antigen/protein recognition unit directly onto moist PVDF membrane in 0.5 ul volumes, diluted in coating buffer. Leave for 1 hour RT in humidified container.

Nitro-cellulose slide-Oncyte slides (Grace Biolabs) are prepared by spotting a solution containing antigen/protein recognition unit directly onto the slide in 0.5 ul volumes, diluted in coating buffer. Leave for 1 hour RT.

Custom glass slides with various coatings and wells for the antibody/protein recognition unit spots cut into the glass may also be used.

### 5.2 Protein Lysate Preparation from Tissue

Lysates made from 3 treatments of mouse livers:

1. livers treated with Azathioprine 100 mg/kg 5 days.

2. chlorambucil i.p. 15 mg/kg 5 days this is the positive control as there is observed liver damage in these tissues seen by clinical pathology.

3. untreated livers.

All these tissues sampled at day 6.

Tissue prepared by adding 1 ml of lysis buffer (100 mM potassium phosphate pH7.8, 0.2% & 1% Triton X-100, 1 mM DTT, 0.2 mM PMSF and 5 ug/ml leupeptin) to 100-150 mg of ground tissue. Ribolyse in Hybaid Ribolyser for 2×10 second bursts on speed 6, microfuge for 5 min 13000 rpm. Remove supernatant from tube, aliquot and perform protein quantification assay. Estimation of protein content is done by the microtiter plate method for Bicinchoninic Acid Assay (BCA). BCA is used as Triton interferes with other protein assays such as Bradford Protein assay.

### 5.3 Assay (I)

Protein mix of interest is labelled with a fluorescent dye (e.g. molecular probes Alexa 488 protein labeling kit). The reaction is stopped using hydroxylamine and excess dye is removed by column separation. The fluorescently labelled protein is then added to PRU's attached to a surface. The position of binding and thus identity of the protein can then be identified. The protein mixture can be “spiked” with a protein which is an antigen for a PRU on the grid this then acts as a positive control to ensure binding has occurred.

### Preparation of Protein Mixture

Make protein mixes, typically a lysate with a positive control “spike” e.g. a protein which is the antigen to an antibody spotted on the grid. To label the protein mix with most molecular probes dyes the total concentration of the mix needs to be ˜2 mg/ml. Label protein mix and controls (spikes) with dyes (Molecular probes) using Alexa™ Protein Labeling Kit Protocol or FluoReporter BODIPY FL Protein Labeling Kit—this protocol was adapted for BODIPY 630/650 and BODIPY 650/665 as follows:

Add 20 ul of Dye to 200 ul lysate conc˜1 mg.

Leave in the dark for 1 hour gently inverting occasionally. Add 7 ul of prepared hydroxylamine, pH 8.5 and leave for 30 min. Place in a spin column, containing 30,000 MW size exclusion resin in PBS (BIORAD) for 3 mins 11000 g. If any precipitate spin for 5 minutes and only take supernatant.

### Binding of Proteins to a PRU Array

All buffers added 20 mls per slide.

Wash slides with 0.05% Tween/PBS buffer for 5 minutes

To saturate non specific protein binding sites block with Blocking buffer for 1 hour

Wash in 0.05% Tween/PBS buffer

SCAN with Scanner Array (Molecular Dynamics)—keep slides from drying out these are used as a background control. Due to drying out separate slides have to be made as controls for grids prepared on PVDF or nitro-cellulose.

Add 100 ul dye+protein mixture to slides and leave in dark for 1 hour, keep in the dark as much as possible from this point.

Wash 2× for 5 mins in TBST Wash Buffer

Wash 1× TBS Wash Buffer remove Tween

Add a couple of drops of Vectashield (Vector laboratories) to nitrocellulose slides (Grace Biolabs) this is to get a refractive index close to that of glass.

Place membrane on a glass slide and cover with a coverslip if using PVDF membrane.

SCAN with Scanner Array (MD)

Buffers:

Carbonate Buffer pH 9.5, 0.2M per litre:

| | | | |
|---|---|---|---|
| NaHCO₃ (Sigma) | 10.5 | g | |
| Na₂CO₃ (Sigma) | 5.16 | g | 1 L. with distilled water. |

Or Carbonate-Bicarbonate buffer capsules 0.05M buffer, pH 9.6. (Sigma)
TBS Wash Buffer pH 8.0 per litre:

| | | | |
|---|---|---|---|
| 0.5M Tris-HCL (Sigma) | 20 | ml | pH 8 |
| 0.15M NaCl (Sigma) | 8.8 | g | 1 L. with distilled water. |

Dissolve the components in 800 ml of deionized water. Adjust to pH 8.0
and bring final volume to 1 L.
TBST Wash Buffer pH 8.0 per litre:

| | | | |
|---|---|---|---|
| 0.5M Tris-HCL (Sigma) | 20 | ml | pH 8 |
| 0.15M NaCl (Sigma) | 8.8 | g | |
| 0.05% Tween-20 (Sigma) | | | 1 L. with distilled water. |

Blocking buffer-make fresh:
1x PBS (Sigma)
0.2% (w/v) Marvel-dried skimmed milk powder
0.1% (w/v) Tween 20 (Sigma)

Lysis buffer:—This buffer does not contain Tris (Tris contains a primary amine and most of the molecular probe dyes react with any primary amine).

100 mM potassium phosphate pH7.8 (Sigma)

0.2% Triton X-1008 (Sigma)

1 mM DTT (Sigma) add just before use

0.2 mM PMSF (Sigma) made up in isopropanol (Sigma) add just before use

5 ug/ml leupeptin (Sigma) add just before use.

The slides are scanned at this first stage to determine background fluorescence of the surface (silane/glutaraldehyde, nitro-cellulose or PVDF) and of the protein recognition units. Background fluorescence can be limited by optimising the concentration of the protein recognition units and also by choosing a dye which is in a range where there is least fluorescence of the protein recognition units.

Dyes were chosen by considering their level of binding to a protein mix and looking at their * Absorption (Abs) and fluorescence emission (Em) maxima. Spectra are detailed in Molecular Probes literature

Dyes are chosen to give minimal background fluorescence and with an emmission spectra suitable for the chosen apparatus of detection.

An Array Scanner-Generation III (Molecular Dynamics) was used:

| | | | |
|---|---|---|---|
| 532_multiples: | excitation green 532 nm | R_1:575B | emission |
| | | R_2:675B | ″ |
| | | R_3:620L | ″ |
| | | R_4:720L | ″ |
| 633_multiples: | excitation red 633 nm | as above | |

Also a Storm (Molecular Dynamics) can be used for membranes only.

| Dye | Abs* (nm) | Em* (nm) |
|---|---|---|
| Alexa 488 | 491 | 515 |
| Alexa 532 | 523 | 548 |
| Alexa 546 | 553 | 569 |
| Alexa 568 | 573 | 596 |
| Alexa 594 | 585 | 610 |
| Bodipy 630/650 | 630 | 650 |
| Bodipy 650/665 | 650 | 670 |

### Results

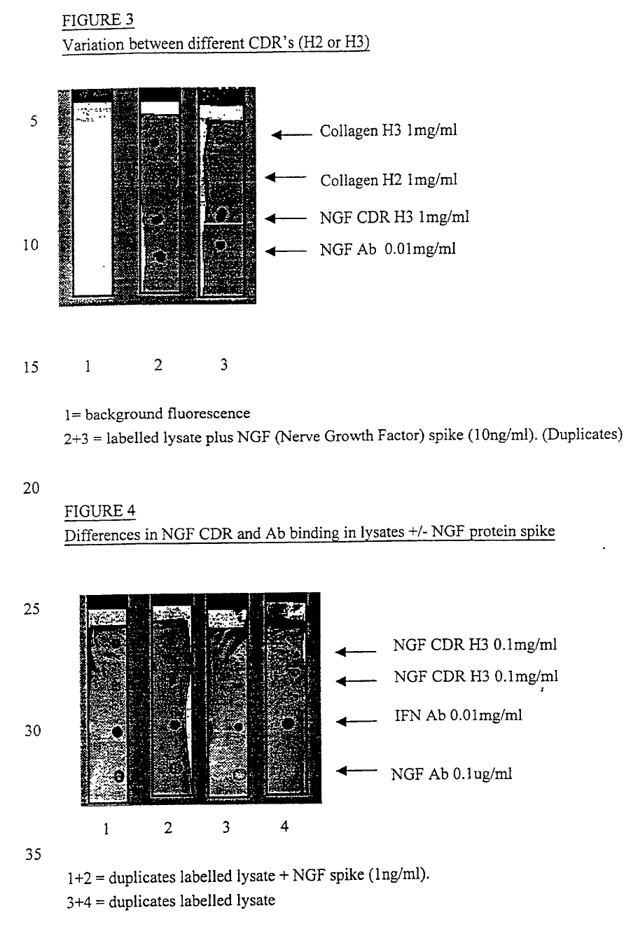

FIG. 3 show the Variation between different CDR's (H2 or H3).

Collagen CDR H3 binds to collagen in the lysate and can be detected. Collagen CDR H2 shows no binding and NGF CDR H3 binds as strongly to the spiked lysate as NGF antibody (0.01 mg/ml).

FIG. 4 shows Differences in NGF CDR and Ab binding in lysates+/−NGF protein spike.

This shows binding by NGF CDR's and Ab in lysates with and without a NGF protein spike.

This experiment demonstrates the use of PRU arrays in detecting proteins in a mixture. The array can be built up with more PRU's relevant to the tissue or treatment of interest, in this case the assay is performed using lysates prepared from treated and untreated animals (e.g. mice treated with Azathioprine and control mice) to determine protein changes that occur when the animal is subjected to a treatment.

### 5.4 Assay (II)

Biotin/avidin method: A method of noncovalent conjugation is to make use of the natural strong binding of avidin for the small biotin molecule. Each avidin molecule contains a maximum of four biotin-binding sites, the interaction can be used to enhance the signal strength in the system of Assay (II).

Roche biotin labeling kit contains modification reagent that can add a functional biotin group to proteins. Biotin-labelled reagents are normally coupled to proteins through free amino groups. Biotin-labelled reagents are detected and quantitated by using avidin or streptavidin. These proteins bind tightly to biotin forming an essentially irreversible complex (Kₐ 10¹⁴ mol⁻¹). NeutrAvidin biotin-binding protein is a protein that has been processed to remove the carbohydrate and lower its isoelectric point- this can sometimes reduce background staining.

Method: Label the protein mix of interest with Biotin using Biotin protein labelling kit (Roche) and pass though a column to remove excess biotin. Bind the protein-mix-biotin to the PRU slide for 1 hour at room temperature (RT). Wash off excess. Wash slide with Streptavidin-dye e.g. (NeutrAvidin™-Alexa 488 conjugate (Molecular Probes) or Streptavidin, Alexa 546 conjugate (Molecular Probes)) for 1 hour RT.

Wash and SCAN.

1. A method for analyzing the protein or peptide content of a mixture comprising exposing a spatial array of protein recognition units to the protein or peptide containing mixture, and detecting the binding of proteins or peptides to the protein recognition units.

2. A method of determining the effect of an environmental factor on protein expression in a target cell, tissue or biological organism, said method comprising the steps of:
(a) providing a plurality of identical spatial arrays of protein recognition units;
(b) exposing the target to an environmental factor for a sufficient time to affect protein expression in said target;
(c) extracting and isolating a protein or peptide containing mixture from said exposed target of step (b);
(d) extracting and isolating a control protein or peptide containing mixture from a target not exposed to said environmental factor;
(e) exposing a spatial array of protein recognition units to the protein or peptide containing mixture from the exposed target, and detecting the binding of proteins or peptides to the protein recognition units;
(f) exposing a spatial array of protein recognition units to the protein or peptide containing mixture from the control target, and detecting the binding of proteins or peptides to the protein recognition units; and
(g) comparing the first and second binding patterns detected in steps (e) and (f) to identify any change in said first pattern from the control pattern, indicative of an effect on protein expression in the target exposed to said environmental factor.

3. The method according to claim 2 wherein said exposing step (b) comprises:
a) administering a transgene into said target;
b) eliminating a gene from said target;
c) administering an exogenous compound to said target;.
d) administering an endogenous compound or an analogue thereof to said target; or
e) exposing said target to a pathogenic microorganism.

4. A method of predicting the toxic effect of a selected test compound on a mammalian subject, said method comprising the steps of:
(a) performing the method of claim 2 or 3 by calibrating the spatial arrays of protein recognition units with a plurality of known and structurally distinct toxicants having a common known toxic effect on mammalian subjects and generating a common “fingerprint” binding pattern characteristic of said common toxic effect;
(b) screening a test compound according to the method of claim 2, steps (a)-(e), to generate a test compound binding pattern; and
(c) comparing the binding pattern of the test compound to said fingerprint binding pattern,
wherein substantial identity between the fingerprint and test patterns indicates that said test compound shares said common toxic effect.

5. The method according to any one of the preceding claims wherein the spatial array comprises protein recognition units immobilized on a solid or semi-solid support.

6. The method according to any one of the preceding claims wherein the protein recognition units are selected from minimal recognition units, antibodies or antibody fragments, high affinity peptides and complementarity determining regions.

7. The method according to claim 6 wherein the protein recognition units are complementarity determining regions.

8. A method as claimed in any one of the preceding claims wherein the detection of binding is achieved using a staining method or using radiolabelling or chemical labeling, the labeling being performed either before or after the binding step.

9. A method according to any one of the preceding claims wherein detection of binding comprises scanning the spatial array using an optical or laser scanner and determining the co-ordinates of binding activity by a computing means.

10. The method according to any one of the preceding claims wherein the protein or peptide containing mixture is a cell extract isolated from a biological organism.

11. A spatial array of complementarity determining regions for use in the method according to any one of the preceding claims.
